(19) United States
(12) Patent Application Publication
(10) Pub. No.: US 2006/0257915 A1
(43) Pub. Date: Nov. 16, 2006

(54) Methods of producing competitive aptamer fret reagents and assays

(75) Inventors: John G. Bruno, San Antonio, TX (US); Joseph Chanpong, San Antonio, TX (US)

Correspondence Address: LOEFFLER JONAS & TUGGEY, LLP, 755 EAST MULBERRY STREET, SUITE 200, SAN ANTONIO, TX, 78212, US

(73) Assignee: PRONUCLEOTEIN BIOTECHNOLOGIES, LLC

(21) Appl. No.: 11/433,283

(22) Filed: May 12, 2006

Related U.S. Application Data
(60) Provisional application No. 60/681,084, filed on May 13, 2005.

(51) Int. Cl. C12Q 1/68

(52) U.S. Cl. 435/6

(57) ABSTRACT

Methods are described for the production and use of fluorescence resonance energy transfer (FRET)-based competitive displacement aptamer assay formats. The assay schemes involve FRET in which the analyte (target) is quencher (Q)-labeled and previously bound by a fluorophore (F)-labeled aptamer such that when unlabeled analyte is added to the system and excited by specific wavelengths of light, the fluorescence intensity of the system changes in proportion to the amount of unlabeled analyte added. Alternatively, the aptamer can be Q-labeled and previously bound to an F-labeled analyte so that when unlabeled analyte enters the system, the fluorescence intensity also changes in proportion to the amount of unlabeled analyte. The F or Q is covalently linked to nucleotide triphosphates (NTPs), which are incorporated into the aptamer by various nucleic acid polymerases, such as Taq during PCR, and then selected by affinity chromatography, size-exclusion, and fluorescence techniques.

This application is based upon and claims priority from U.S. Provisional application Ser. No. 60/681,084, which is incorporated herein by reference.

### BACKGROUND OF THE INVENTION

1. Field of the Invention

The present invention relates to the field of aptamer- and nucleic acid-based diagnostics. More particularly, it relates to methods for the production and use of fluorescence resonance energy transfer (“FRET”) DNA or RNA aptamers for competitive displacement aptamer assay formats. The present invention provides for aptamer-related FRET assay schemes involving competitive displacement formats in which the aptamer contains fluorophores (“F”) (is F-labeled) and the target contains quenchers (“Q”) (is Q-labeled), or vice versa. The aptamer can be F-labeled or Q-labeled by incorporation of the F or Q derivatives of nucleotide triphosphates. Incorporation may be accomplished by simple chemical conjugations through bifunctional linkers, or key functional groups such as aldehydes, carbodiimides, carboxyls, N-hydroxy-succinimide (NHS) esters, thiols, etc.

2. Background Information

Competitive displacement aptamer-FRET is a new class of assay desirable for its use in rapid (within minutes), one-step, homogeneous assays involving no wash steps (simple bind and detect quantitative assays). Others have described FRET-aptamer methods for various target analytes that consist of placing the F and Q moieties either on the 5′ and 3′ ends respectively to act like a “molecular (aptamer) beacon” or placing only F in the heart of the aptamer structure to be “quenched” by another proximal F or the DNA or RNA itself. These preceding FRET-aptamer methods are all highly engineered and based on some prior knowledge of particular aptamer sequences and secondary structures, thereby enabling clues as to where F might be placed in order to optimize FRET results.

### SUMMARY OF THE INVENTION

The nucleic acid-based “molecular beacons” snap open upon binding to an analyte or upon hybridizing to a complementary sequence, but beacons have always been end-labeled with F and Q at the 3′ and 5′ ends. The present invention provides that F-labeled or Q-labeled aptamers may be labeled anywhere in their structure that places the F or Q within the Forster distance of approximately 60-85 Angstroms of the corresponding F or Q on the labeled target analyte to achieve quenching prior to or after target analyte binding to the aptamer “binding pocket” (typically a “loop” in the secondary structure). The F and Q molecules used can include any number of appropriate fluorophores and quenchers as long as they are spectrally matched so the emission spectrum of F overlaps significantly (almost completely) with the absorption spectrum of Q.

A process in which F and Q are incorporated into an aptamer population is generally referred to as “doping.” The present invention provides a new method for natural selection of F-labeled or Q-labeled aptamers that contain F-NTPs or Q-NTPs in the heart of an aptamer binding loop or pocket by PCR or other enzymatic means. The present invention describes a type of aptamer in which F and Q are incorporated into an aptamer population via their nucleotide triphosphate derivatives (for example, Alexfluor™-NTPs, Cascade Blue®-NTPs, Chromatide®-NTPs, fluorescein-NTPs, rhodamine-NTPs, Rhodamine Green™-NTPs, tetramethylrhodamine-dNTPs, Oregon Green®-NTPs, and Texas Red®-NTPs may be used to provide the fluorophores, while dabcyl-NTPs, Black Hole Quencher or BHQ™-NTPs, and QSY™ dye-NTPs may be used for the quenchers) by PCR after several rounds of selection and amplification without the F- and Q-modified bases. The advantage of this F or Q “doping” method is two-fold: 1) the method allows nature to take its course and select the most sensitive F-labeled or Q-labeled aptamer target interactions in solution, and 2) the positions of F or Q within the aptamer structure can be determined via exonuclease digestion of the F-labeled or Q-labeled aptamer followed by mass spectral analysis of the resulting fragments, thereby eliminating the need to “engineer” the F or Q moieties into a prospective aptamer binding pocket or loop. Sequence and mass spectral data can be used to further optimize the competitive aptamer-FRET assay performance after natural selection as well.

If the target molecule is a larger water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTPs that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule (generally defined as a molecule with molecular weight of ≦1,000 Daltons), then exposure of the nascent F-labeled and Q-labeled DNA or RNA random library to the target is done by immobilizing the target. The small molecule can be immobilized on a column, membrane, plastic or glass bead, magnetic bead, or other matrix. If no functional group is available on the small molecule for immobilization, the target can be immobilized by the Mannich reaction (formaldehyde-based condensation reaction) on a device similar to a PharmaLink™ column. Elution of bound DNA from the small molecule affinity column, membrane, beads or other matrix by use of 0.2-3.0M sodium acetate at a pH ranging between 3 and 7, although the optimal pH is approximately 5.2.

The candidate FRET-aptamers are separated based on physical properties such as charge or weak interactions by various types of high performance liquid chromatography (“HPLC”), digested at each end with specific exonucleases (snake venom phosphodiesterase on the 3′ end and calf spleen phosphodiesterase on the 5′ end). The resulting oligonucleotide fragments, each one base shorter than the predecessor, are subjected to mass spectral analysis which can reveal the nucleotide sequences as well as the positions of F and Q within the FRET-aptamers. Once the FRET-aptamer sequence is known with the positions of F and Q, it can be further manipulated during solid-phase DNA or RNA synthesis in an attempt to make the FRET assay more sensitive and specific.

The competitive displacement aptamer-FRET assay format of the present invention is unique. The competitive format generally requires a lower affinity aptamer in order to be able to release the F-labeled or Q-labeled target analyte and allow competition for the binding site. This may lead to less sensitivity in some assays.

### BRIEF DESCRIPTION OF THE DRAWINGS

FIG. 1. is a schematic illustration that illustrates a comparison of possible nucleic acid FRET assay formats.

FIG. 2. are line graphs mapping relative fluorescence intensity against the concentration of surface protein.

### DETAILED DESCRIPTION OF THE PREFERRED EMBODIMENT

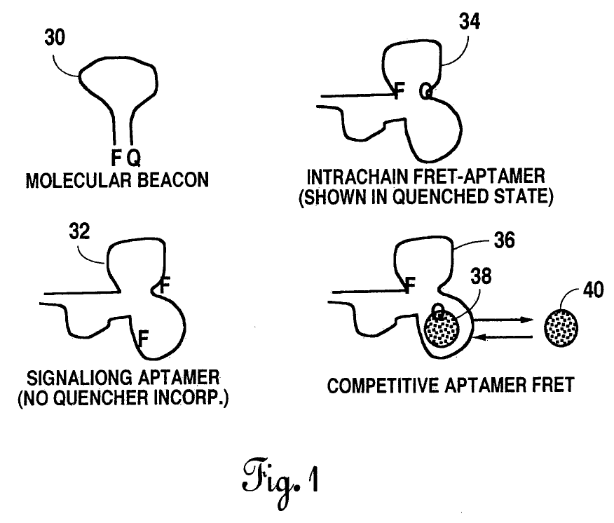

Referring to the figures, FIG. 1. provides a comparison of possible nucleic acid FRET assay formats. It illustrates how the competitive aptamer-FRET scheme differs from other oligonucleotide-based FRET assay formats. Upper left is a molecular beacon (10) which may or may not be an aptamer, but is typically a short oligonucleotide used to hybridize to other DNA or RNA molecules and exhibit FRET upon hybridizing. Molecular beacons are only labeled with F and Q at the ends of the DNA molecule. Lower left is a signaling aptamer (12), which does not contain a quencher molecule, but relies upon fluorophore self-quenching or weak intrinsic quenching of the DNA or RNA to achieve limited FRET. Upper right is an intrachain FRET-aptamer (14) containing F and Q molecules built into the interior structure of the aptamer. Intrachain FRET-aptamers are naturally selected and characterized by the processes described herein. Lower right shows a competitive aptamer-FRET (16) motif in which the aptamer contains either F or Q and the target molecule (18) is labeled with the complementary F or Q. Introduction of unlabeled target molecules (20) then shifts the equilibrium so that some labeled target molecules (18) are liberated from the labeled aptamer (16) and modulate the fluorescence level of the solution up or down thereby achieving FRET. A target analyte (20) is either unlabeled or labeled with a quencher (Q). F and Q can be switched from placement in the aptamer (16) to placement in the target analyte (20) and vice versa.

F-labeled or Q-labeled aptamers (labeled by the polymerase chain reaction (PCR) or other enzymatic incorporation of F-NTPs or Q-NTPs) may be used in competitive or displacement type assays in which the fluorescence light levels change proportionately in response to the addition of various levels of unlabeled analyte which compete to bind with the F-labeled or Q-labeled analytes.

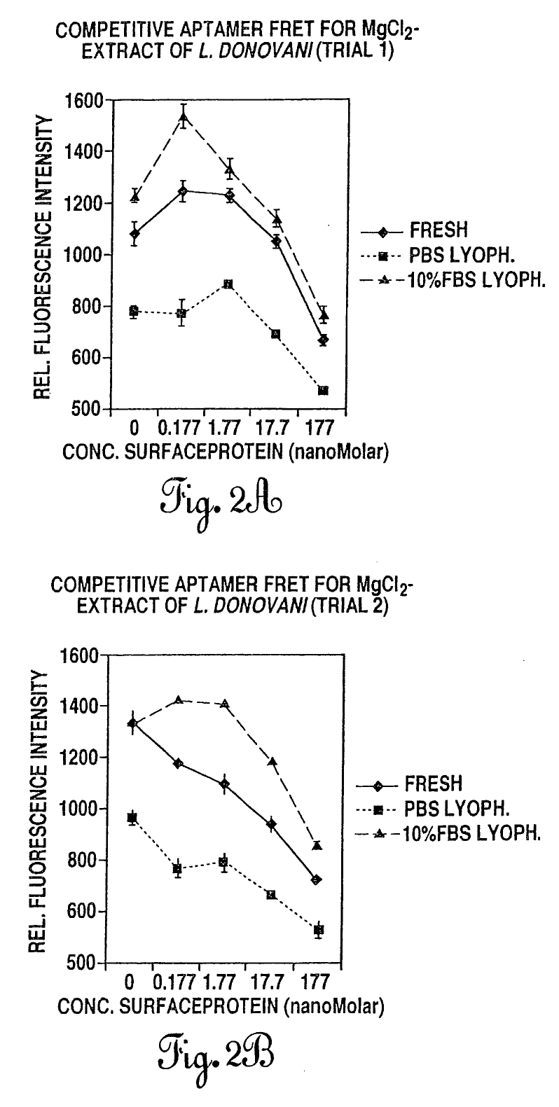

Competitive aptamer-FRET assays may be used for the detection and quantitation of small molecules (<1,000 Daltons) including pesticides, natural and synthetic amino acids and their derivatives (e.g., histidine, histamine, homocysteine, DOPA, melatonin, nitrotyrosine, etc.), short chain proteolysis products such as cadaverine, putrescine, the polyamines spermine and spermidine, nitrogen bases of DNA or RNA, nucleosides, nucleotides, and their cyclical isoforms (e.g., cAMP and cGMP), cellular metabolites (e.g., urea, uric acid), pharmaceuticals (therapeutic drugs), drugs of abuse (e.g., narcotics, hallucinogens, gamma-hydroxybutyrate, etc.), cellular mediators (e.g., cytokines, chemokines, immune modulators, neural modulators, inflammatory modulators such as prostaglandins, etc.), or their metabolites, explosives (e.g., trinitrotoluene) and their breakdown products or byproducts, peptides and their derivatives, macromolecules including proteins (such as bacterial surface proteins from Leishmania donovani, See FIGS. 2A and 2B), glycoproteins, lipids, glycolipids, nucleic acids, polysaccharides, lipopolysaccharides, etc.), whole cells, and subcellular organelles or cellular fractions.

If the target molecule is a larger water-soluble molecule such as a protein, glycoprotein, or other water soluble macromolecule, then exposure of the nascent F-labeled and Q-labeled DNA or RNA random library to the free target analyte is done in solution. If the target is a soluble protein or other larger water-soluble molecule, then the optimal FRET-aptamer-target complexes are separated by size-exclusion chromatography. The FRET-aptamer-target complex population of molecules is the heaviest subset in solution and will emerge from a size-exclusion column first, followed by unbound FRET-aptamers and unbound proteins or other targets. Among the subset of analyte-bound aptamers there will be heterogeneity in the numbers of F- and Q-NTPs that are incorporated as well as nucleotide sequence differences, which will again effect the mass, electrical charge, and weak interaction capabilities (e.g., hydrophobicity and hydrophilicity) of each analyte-aptamer complex. These differences in physical properties of the aptamer-analyte complexes can then be used to separate out or partition the bound from unbound analyte-aptamer complexes.

If the target is a small molecule, then exposure of the nascent F-labeled and Q-labeled DNA or RNA random library to the target may be done by immobilizing the target. The small molecule can be immobilized on a column, membrane, plastic or glass bead, magnetic bead, or other matrix. If no functional group is available on the small molecule for immobilization, the target can be immobilized by the Mannich reaction (formaldehyde-based condensation reaction) on a PharmaLink™ column. Elution of bound DNA from the small molecule affinity column, membrane, beads or other matrix by use of 0.2-3.0M sodium acetate at a pH ranging between 3 and 7, although the optimal pH is approximately 5.2.

These can be separated from the non-binding doped DNA molecules by running the aptamer-protein aggregates (or selected aptamers-protein aggregates) through a size-exclusion column, by means of size-exclusion chromatography using Sephadex™ or other gel materials in the column. Since they vary in weight due to variations in aptamers sequences and degree of labeling, they can be separated into fractions with different fluorescence intensities. Purification methods such as preparative gel electrophoresis are possible as well. Small volume fractions (≦1 mL) can be collected from the column and analyzed for absorbance at 260 nm and 280 nm which are characteristic wavelengths for DNA and proteins. The heaviest materials come through a size-exclusion column first. Therefore, the DNA-protein complexes will come out of the column before either the DNA or protein alone.

Means of separating FRET-aptamer-target complexes from solution by alternate techniques (other than size-exclusion chromatography) include, without limitation, molecular weight cut off spin columns, dialysis, gel electrophoresis, thin layer chromatography (TLC), and differential centrifugation using density gradient materials.

The optimal (most sensitive or highest signal to noise ratio) FRET-aptamers among the bound class of FRET-aptamer-target complexes are identified by assessment of fluorescence intensity for various fractions of the FRET-aptamer-target class. The separated DNA-protein complexes will exhibit the highest absorbance at established wavelengths, such as 260 nm and 280 nm. The fractions showing the highest absorbance at the given wavelengths, such as 260 nm and 280 nm, are then further analyzed for fluorescence and those fractions exhibiting the greatest fluorescence are selected for separation and sequencing.

These similar FRET-aptamers may be further separated using techniques such as ion pair reverse-phase high performance liquid chromatography, ion-exchange chromatography (IEC, either low pressure or HPLC versions of IEC), thin layer chromatography (TLC), capillary electrophoresis, or similar techniques.

The final FRET-aptamers are able to act as one-step “lights on” or “lights off” binding and detection components in assays.

Competitive FRET-aptamers that are to be used in assays with long shelf-lives may be lyophilized (freeze dried) and then later reconstituted.

FIGS. 2A and 2B. are line graphs mapping the fluorescence intensity of the DNA aptamers against the concentration of the surface protein. The figures present results from two independent trials of a competitive aptamer-FRET assay involving fluorophore-labeled DNA aptamers and surface extracted proteins from Leishmania donovani bacteria. In this type of assay, the fluorescence intensity decreases as a function of increasing analyte concentration, and is thus referred to as a “lights off” assay. If the fluorescence intensity increases as a function of increasing analyte concentration, then it is referred to as a “lights on” assay. Also shown are translations of the assay curve up or down due to lyophilization (freeze-drying) in the absence or presence of 10% fetal bovine serum (FBS). Error bars represent the standard deviations of the mean for three measurements.

### EXAMPLE 1

Competitive Aptamer-FRET Assay for Surface Proteins Extracted from Bacteria (L. donovani).

In this example, surface proteins from heat-killed Leishmania donovani were extracted with 3 M MgCl₂ overnight at 4° C. These proteins were then linked to tosyl-magnetic microbeads and used in a standard SELEX aptamer generation protocol. After 5 rounds of SELEX, the aptamer population was “doped” during the standard PCR reaction with 3 uM fluorescein-dUTP and purified on 10 kD molecular weight cut off spin columns. Some of the L. donovani surface proteins were then labeled with dabcyl-NHS ester and purified on a PD-10 (Sephadex G25) column. The dabcyl-labeled surface proteins were combined with the fluorescein-labeled aptamer population so as to produce a 1:1 fluorescein-aptamer:dabcyl-protein ratio. Thereafter, unlabeled L. donovani surface proteins were introduced into the assay system to compete with the labeled proteins for binding to the aptamers, thereby producing the “lights off” FRET assay results depicted in FIGS. 2A and 2B (fresh assay results, solid line). The assays were also examined following lyophilization and reconstitution of the FRET-aptamers in the presence or absence of 10% fetal bovine serum (FBS) as a possible preservative with the results shown in FIGS. 2A and 2B.

Although the invention has been described with reference to specific embodiments, this description is not meant to be construed in a limited sense. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

What is claimed:

1. A method of using a competitive type assay, comprising:
running an assay;
incorporating F-labeled or Q-labeled aptamers, wherein said aptamers are labeled with said F's and Q's located on the interior portion of said aptamer;
adding a volume of unlabeled analyte, wherein said analyte competes to bind with said F-labeled or Q-labeled analytes; and
wherein fluorescence light levels change proportionately in response to the amount of said volume of unlabeled analyte.

2. The method of claim 1, wherein said competitive type assay is used the detection and quantitation of small molecules.

3. The method of claim 2, wherein said small molecules are less than 1,000 Daltons.

4. The method of claim 2, wherein said small molecules are selected from the group consisting of pesticides, natural and synthetic amino acids and their derivatives, histidine, histamine, homocysteine, DOPA, melatonin, nitrotyrosine, short chain proteolysis products, cadaverine, putrescine, polyamines, spermine, spermidine, nitrogen bases of DNA or RNA, nucleosides, nucleotides, nucleotide cyclical isoforms, cAMP, cGMP, cellular metabolites, urea, uric acid, pharmaceuticals, therapeutic drugs, illegal drugs, narcotics, hallucinogens, gamma-hydroxybutyrate, cellular mediators, cytokines, chemokines, immune modulators, neural modulators, inflammatory modulators, prostaglandins, prostaglandin metabolites, explosives, trinitrotoluene, explosive breakdown products or byproducts, peptides and their derivatives, macromolecules, proteins, bacterial surface proteins, glycoproteins, lipids, glycolipids, nucleic acids, polysaccharides, lipopolysaccharides, whole cells, and subcellular organelles or cellular fractions.

5. The method of claim 1, wherein said fluorophores are selected from the group consisting of Alexfluor™-NTPs, Cascade Blue®-NTPs, Chromatide®-NTPs, fluorescein-NTPs, rhodamine-NTPs, Rhodamine Green™-NTPs, tetramethylrhodamine-dNTPs, Oregon Green®-NTPs, and Texas Red®-NTPs.

6. The method of claim 1, wherein said quenchers are selected from the group consisting of dabcyl-NTPs, Black Hole Quencher or BHQ™-NTPs, and QSY™ dye-NTPs.

7. The method of claim 2, wherein said fluorophores are selected from the group consisting of Alexfluor™-NTPs, Cascade Blue®-NTPs, Chromatide®-NTPs, fluorescein-NTPs, rhodamine-NTPs, Rhodamine Green™-NTPs, tetramethylrhodamine-dNTPs, Oregon Green®-NTPs, and Texas Red®-NTPs.

8. The method of claim 2, wherein said quenchers are selected from the group consisting of dabcyl-NTPs, Black Hole Quencher or BHQ™-NTPs, and QSY™ dye-NTPs.

9. The method of claim 2, further comprising immobilizing said small molecules.

10. The method of claim 9, wherein said immobilizing step is accomplished on a column, membrane, plastic or glass bead, magnetic bead, or other matrix.

11. The method of claim 10, further comprising eluting bound aptamers from said column, membrane, plastic or glass bead, magnetic bead, or other matrix by use of 0.2-3.0M sodium acetate at a pH of between 3 and 7.

12. The method of claim 10, further comprising eluting bound aptamers from said column, membrane, plastic or glass bead, magnetic bead, or other matrix by use of 0.2-3.0M sodium acetate at a pH of 5.2.

13. The method of claim 9, wherein said immobilizing step is accomplished via a formaldehyde-based condensation reaction.

14. The method of claim 2, wherein if said target molecules are larger water-soluble molecule such as a protein, glycoprotein, or other water soluble macromolecule, then said exposing step is accomplished in solution.

15. The method of claim 14, wherein said first separating step is accomplished via one of size-exclusion chromatography, molecular weight cut off spin columns, dialysis, gel electrophoresis, thin layer chromatography (TLC), or differential centrifugation using density gradient materials.

16. The method of claim 2, further comprising identifying optimal bound FRET-aptamers via fluorescence intensity.

17. The method of claim 16, further comprising separating said optimal bound FRET-aptamers via ion pair reverse-phase high performance liquid chromatography, ion-exchange chromatography, thin layer chromatography, capillary electrophoresis, or similar techniques.

18. The method of claim 17, further comprising:
digestion, in a first digesting step, the sequences and structures of said unbound FRET-aptamer using snake venom phosphodiesterase exonuclease of the 3′ end of said unbound FRET-aptamer to generate oligonucleotide fragments;
digestion, in a second digesting step, the sequences and structures of said unbound FRET-aptamer using calf spleen phosphodiesterase of the 5′ end of said unbound FRET-aptamer to generate oligonucleotide fragments;
performing mass spectral analysis of said oligonucleotide fragments; and
determining the nucleotide sequences and placement of F and Q moieties of said oligonucleotide fragments.

19. The method of claim 2, wherein said FRET-aptamers are for use in assays with long shelf-lives, said method further comprising:
lyophilization of said competitive FRET-aptamers; and
reconstitution of said competitive FRET-aptamers.
